(19) United States
(12) Patent Application Publication
(10) Pub. No.: US 2004/0115632 A1
(43) Pub. Date: Jun. 17, 2004

(54) Methods and reaction mixture reagent for increasing the 3' end specificity of oligonucleotide priming

(76) Inventor: Martin Eduardo Mautner, Buenos Aires (AR)

Correspondence Address: WILDMAN, HARROLD, ALLEN & DIXON, 225 WEST WACKER DRIVE, CHICAGO, IL, 60606, US

(21) Appl. No.: 10/316,745

(22) Filed: Dec. 11, 2002

(51) Int. Cl. C12Q 1/68; C12P 19/34

(52) U.S. Cl. 435/6; 435/91.2

(57) ABSTRACT

The invention provides a method for reducing the efficiency of primer extension by polymerase enzymes when the 3′ end of a primer does not hybridize perfectly with the target, increasing the selectivity of single nucleotide mutation or gene analyses by suppressing false positive results, comprising the steps of: (a) obtaining a nucleic acid sample; (b) hybridizing said nucleic acid sample to a primer; (c) subjecting said nucleic acid sample hybridized to a extension reaction by extending a primer with a polymerizing enzyme, wherein the reaction extension mixture medium contains an intercalating agent; and (d) detecting the presence of extension products. The intercalating agent may be any intercalating agent as ethidium bromide, dihydroethidium, ethidium homodimer-1, ethidium homodimer-2, acridine, propidium iodide, YOYO®-1 and TOTO®-1. When the intercalating agent is ethidium bromide the concentration is about 4 to 7 μg/ml, preferable 5 μg/ml.

The invention also provides a reaction extension mixture reagent, wherein the reagent comprises: a polymerizing enzyme, dNTPs, a buffer, an intercalating agent as for example ethidium bromide. The reaction extension mixture reagent may be a PCR reaction mixture or anyone medium which an extension reaction of nucleic acid was made.

### BACKGROUND OF THE INVENTION

1. Field of the Invention

The present invention relates to the field of molecular biology. More specifically, the invention is in the field of assays that utilize oligonucleotides as primers in extension reactions. The method of the invention is useful to increase the specificity of PCR or other assays that employ an extension step, when there is a mismatch in the primer 3′ end.

2. Description of the Prior Art

Detecting and identifying variations in DNA sequences among individuals and species has provided insights into evolutionary relationships, inherited disorders, acquired disorders and others aspects of molecular genetic and medicine.

These variations may involve different lengths of DNA, from several nucleotides down to just a single one. The detection of single nucleotide polymorphisms (SNPs) is a challenging task aimed to provide new developments in the field of Molecular Biology.

The analysis of sequence variation has traditionally been performed by restriction fragment length polymorphism (RFLP) in a Southern blot format, or more recently, by digesting PCR products. The RFLP analyses are based on a change in the restriction fragment length as a result of a change in the sequence. Nowadays most techniques rely on the differential annealing of allele-specific oligonucleotides to a template. Some of these techniques are allele-specific oligonucleotide hybridization (ASO), reverse dot blot, competitive oligonucleotide priming (COP), primer extension sequence test (PEST), nucleic acid depolymerization (READIT), and amplification refractory mutation system (ARMS), also known as allele-specific PCR (ASP), PCR amplification of specific alleles (PASA) and allele-specific amplification (ASA).

A key aspect of the methods that are based upon oligonuclotide base-pairing is that the allele-specific oligonuclotide must anneal only to the homologous sequence to prevent misleading results. However, this is not always the case with the methods where 3′ mismatches are used to identify the different alleles. Newton et al.(Nucleic Acids Res. 17: 2503, 1898) and Kwok, et al. (Nucleic Acids Res. 18: 999, 1990) report that a 3′ terminal mismatch on the PCR primer produced variable results, making it necessary to add a 3′ terminal mismatch accompanied by a second mismatch within the last four nucleotides of the primer. The arbitrariness in the addition of extra mismatches near the 3′ end on individual primers in every particular instance limits the general application of the technique in a simple and universal fashion. Also, because of the lower selectivity due to the formation of false DNA synthesis products when using single 3′ mismatch primers, the informative power of the gene variation analyses and gene mutation analyses is limited. The formation of false DNA synthesis products can lead to false findings, as a result of which concerning risks arise for the patient and for biomedical research in general.

U.S. Pat. No. 6,403,313 teaches methods to detect specific hybridization between single-stranded probes and non-denatured double-stranded targets to form triplex by an intercalating agent, thus obviating the need to denature the target. This method can be used to determine the number of mismatched pairs in a hybridization complex, and to map genomes.

Bodmer et al, WO 01/75155, teach methods that can distinguish between specific and non-specific amplification products, for example adding to the post-amplification products an amount of small molecules sufficient to increase the pH of the sample products, wherein the pH is 11-14 and then assaying the post-amplification sample product in order to detect and/or quantify any double-stranded nucleic acid present. The method is useful for detecting and/or quantifying a specific double-stranded nucleic acid amplification product in a nucleic amplification reaction post-amplification sample, as in ARMS-PCR methods for SNP typing.

U.S. Pat. No. 5,639,611 discloses an allele specific PCR reaction with two primers (mutant and normal alleles), which one of the primers is complementary to the first allele, but which primer forms a mismatch with the second allele at the 3′ end of the primer, employing a DNA polymerase wherein the first allele is specifically amplified but little or no amplification the second allele occurs.

U.S. patent application Ser. No. 10/009,761 discloses a method for, detecting a single nucleotide polymorphism in a target by isothermal nucleic acid amplification, hybridizing a detector primer to the target wherein the detector primer comprises a diagnostic nucleotide for the single nucleotide polymorphism about one to four nucleotides from 3′ terminal nucleotide of the detector primer, which is complementary to the target sequence, amplifying the target, determining an efficiency of detector primer extension and detecting de presence or absence of the single nucleotide polymorphism based on the efficiency of detector primer extension. This application disclosed the ARMS method.

U.S. Pat. No. 6,312,894 discloses a hybridization and mismatch discrimination using oligonucleotides conjugated to minor grooves binders. The minor grooves binders is a molecule having a molecular weight of approximately 150 to 2,000 Daltons as 1,2-dihydro-(3H)-pyrrolo[3,2-e]indole-7-carboxylate that binds in a non-intercalating manner into de minor groove of a double-stranded nucleic acid.

It would be therefore convenient to have a method to increase the selectivity of gene variation analyses and, by suppressing the formation of false positives, to prevent wrong diagnoses and erroneous findings. The goal of the present invention is increase the selectivity in specific nucleic acids sequence analyses adding an intercalating agent to the conventional reactions medium, reducing the efficiency of primer extension by polymerases when the 3′ end of a primer does not hybridize perfectly with the target sequence. The addition of the intercalating agent avoids the need to place a second mismatch in the sequence of the detector primer which is not directed to detection or identification of the allele of interest.

### SUMMARY OF THE INVENTION

It is therefore an object of the present invention to provide a method for reducing the efficiency of primer extension by polymerase enzymes when the 3′ end of a primer does not hybridize perfectly with the target, increasing the selectivity of single nucleotide mutation or gene analyses by suppressing false positive results, comprising the steps of:

(a) obtaining a nucleic acid sample;

(b) hybridizing said nucleic acid sample to a primer;

(c) subjecting said nucleic acid sample hybridized to a extension reaction by extending the primer with a polymerizing enzyme, wherein the reaction extension mixture medium contains an intercalating agent; and

(d) detecting the presence of extension products.

In a preferred embodiment the methods comprises: (a) obtaining a nucleic acid sample; (b) hybridizing said nucleic acid sample to primer pair by subjecting said nucleic acid sample hybridized to a PCR, wherein the PCR reaction mixture contains an intercalating agent; and (c) detecting the presence of amplification products

The intercalating agent may be any intercalating agent as ethidium bromide, dihydroethidium, ethidium homodimer-1, ethidium homodimer-2, acridine, propidium iodide, YOYO®-1, TOTO®-1, or any other flat organic molecule capable of stacking between the nucleic acid bases. In a preferred embodiment the intercalating agent is ethidium bromide at a concentration about 4 to 7 μg/ml, preferably 5 μg/ml.

The methods of the present invention decrease the amount of extension products when the 3′ end of a primer does not hybridize perfectly with the target sequence, increase the selectivity detection of single nucleotide mutation and/or suppress false positive extension products in gene analyses.

It is still another object of the present invention to provide a reaction extension mixture reagent, wherein the reagent comprises: a polymerizing enzyme, dNTPs, a buffer, an intercalating agent as for example ethidium bromide, dihydroethidium, ethidium homodimer-1, ethidium homodimer-2, acridine, propidium iodide, YOYO®-1, TOTO®-1, or any other flat organic molecule capable of stacking between the nucleic acid bases.

The reaction extension mixture reagent may be a PCR reaction mixture or any one medium which an extension reaction of nucleic acid was made.

The above and other objects, features and advantages of this invention will be better understood when taken in connection with the accompanying drawings and description.

### BRIEF DESCRIPTION OF THE DRAWINGS

The present invention is illustrated by way of example in the following drawings wherein:

FIG. 1 shows the amplification results on mtDNA in 2% agarose gel stained with ethidium bromide carrying a T to C transition at position 16311 using primers FW I (SEQ ID No 1) and either 16311A (SEQ ID No 3), 16311G (SEQ ID No 4), 16311T (SEQ ID No 5) or 16311C (SEQ ID No 6) at different ethidium bromide concentrations.

Lane 1 through 4: 16311A (SEQ ID No 3), 16311G (SEQ ID No 4), 16311T (SEQ ID No 5) and 16311C (SEQ ID No 6) without ethidium bromide. Lane 5 through 8: 16311A (SEQ ID No 3), 16311G (SEQ ID No 4), 16311T (SEQ ID No 5) and 16311C (SEQ ID No 6) with 4.5 ug/ml ethidium bromide. Lane 9 through 12: 16311A (SEQ ID No 3), 16311G (SEQ ID No 4), 16311T (SEQ ID No 5) and 16311C (SEQ ID No 6) with 5.0 ug/ml ethidium bromide. Lane 13 through 16: 16311A (SEQ ID No 3), 16311G (SEQ ID No 4), 16311T (SEQ ID No 5) and 16311C (SEQ ID No 6) C with 5.5 ug/ml ethidium bromide. Lane 16 through 20: 16311A (SEQ ID No 3), 16311G (SEQ ID N° 4), 16311T (SEQ ID No 5) and 16311C (SEQ ID No 6) with 6.0 ug/ml ethidium bromide. Lane 21: 100 bp ladder (Promega Corp.);

FIG. 2 shows the amplification results on mtDNA in 2% agarose gel stained with ethidium bromide showing the amplification results on mtDNA carrying an Andersons' T nucleotide at position 16311 using primers FW I and either 16311A (SEQ ID No 3), 16311G (SEQ ID N° 4), 16311T (SEQ ID No 5) or 16311C (SEQ ID No 6) at different ethidium bromide concentrations.

Lane 1 through 4: 16311A (SEQ ID No 3), 16311G (SEQ ID No 4), 16311T (SEQ ID No 5) and 16311C (SEQ ID No 6) without ethidium bromide. Lane 5 through 8: 16311A (SEQ ID No 3), 16311G (SEQ ID No 4), 16311T (SEQ ID No 5) and 16311C (SEQ ID No 6) with 4.5 ug/ml ethidium bromide. Lane 9 through 12: 16311A (SEQ ID No 3), 16311G (SEQ ID No 4), 16311T (SEQ ID No 5) and 16311C (SEQ ID No 6) with 5.0 ug/ml ethidium bromide. Lane 13 through 16: 16311A (SEQ ID No 3), 16311G (SEQ ID No 4), 16311T (SEQ ID No 5) and 16311C (SEQ ID No 6) with 5.5 ug/ml ethidium bromide. Lane 16 through 20: 16311A (SEQ ID No 3), 16311G (SEQ ID No 4), 16311T (SEQ ID No 5) and 16311C (SEQ ID No 6) with 6.0 ug/ml ethidium bromide. Lane 21: 100 bp ladder (Promega Corp);

FIG. 3 shows the amplification results on mtDNA in a 2% agarose gel stained with ethidium bromide showing the amplification results on mtDNA carrying a C nucleotide at position 16256 using primers FW I and either 16256A (SEQ ID No 7), 16256G (SEQ ID No 8), 16256T (SEQ ID No 9) or 16256C (SEQ ID No 10) at different ethidium bromide concentrations.

Lane 1 through 4: 16256A (SEQ ID No 7), 16256G (SEQ ID No 8), 16256T (SEQ ID No 9) and 16256C (SEQ ID No 10) without ethidium bromide. Lane 5 through 8: 16256A (SEQ ID No 7), 16256G (SEQ ID No 8), 16256T (SEQ ID No 9) and 16256C (SEQ ID No 10) with 4.5 ug/ml ethidium bromide. Lane 9 through 12: 16256A (SEQ ID No 7), 16256G (SEQ ID No 8), 16256T (SEQ ID No 9) and 16256C (SEQ ID No 10) with 5.0 ug/ml ethidium bromide. Lane 13 through 16: 16256A (SEQ ID N° 7), 16256G (SEQ ID N° 8), 16256T (SEQ ID N° 9) and 16256C (SEQ ID No 10) with 5.5 ug/ml ethidium bromide. Lane 16 through 20: 16256A (SEQ ID No 7), 16256G (SEQ ID No 8), 16256T (SEQ ID No 9) and 16256C (SEQ ID No 10) with 6.0 ug/ml ethidium bromide. Lane 21: 100 bp ladder (Promega Corp.);

FIG. 4 shows the amplification results on mtDNA in 2% agarose gel stained with ethidium bromide showing the amplification results on mtDNA carrying a G nucleotide at position 143 using primers FW II (SEQ ID No 2) and either 143A (SEQ ID No 11), 143G (SEQ ID No 12), 143T (SEQ ID No 13) or 143C (SEQ ID No 14) at different ethidium bromide concentrations.

Lane 1 through 4: 143A (SEQ ID N° 11), 143G (SEQ ID No 12), 143T (SEQ ID N° 13) and 143C (SEQ ID No 14) without ethidium bromide. Lane 5 through 8: 143A (SEQ ID No 11), 143G (SEQ ID No 12), 143T (SEQ ID No 13) and 143C (SEQ ID No 14) with 4.5 ug/ml ethidium bromide. Lane 9 through 12: 143A (SEQ ID No 11), 143G (SEQ ID No 12), 143T (SEQ ID No 13) and 143C (SEQ ID No 14) with 5.0 ug/ml ethidium bromide. Lane 13 through 16: 143A (SEQ ID No 11), 143G (SEQ ID No 12), 143T (SEQ ID No 13) and 143C (SEQ ID No 14) with 5.5 ug/ml ethidium bromide. Lane 16 through 20: 143A (SEQ ID No 11), 143G (SEQ ID No 12), 143T (SEQ ID No 13) and 143C (SEQ ID No 14) with 6.0 ug/ml ethidium bromide. Lane 21: 100 bp ladder

(Promega Corp.)

FIG. 5 shows the amplification results on mtDNA in 2% agarose gel stained with ethidium bromide showing the amplification results on mtDNA carrying a T to C transition at position 16311 with 5.0 ug/ml ethidium bromide using primers FW I and either 16311A (SEQ ID No 3) or 16311G (SEQ ID No 4) at different concentrations of downstream primers.

Lane 1 through 2: 100 pmole of 16311A (SEQ ID No 3) and 16311G (SEQ ID No 4) and 5.0 ug/ml ethidium bromide. Lane 3 through 4: 50 pmole 16311A (SEQ ID No 3) and 16311G (SEQ ID No 4), 5.0 ug/ml ethidium bromide. Lane 5 through 6: 5 pmole 16311A (SEQ ID No 3) and 16311G (SEQ ID No 4), 5.0 ug/ml of ethidium bromide. Lane 7 through 8: 2.5 pmole 16311A (SEQ ID No 3) (SEQ ID No 3) and 16311G (SEQ ID No 4), 5.0 ug/ml ethidium bromide. Lane 9 through 10: 0.5 pmole 16311A (SEQ ID No 3), (SEQ ID No 3) and 16311G (SEQ ID No 4), 5.0 ug/ml ethidium bromide. Lane 11: 100 bp ladder (Promega Corp.). Lane 13 through 14: 100 pmole 16311A (SEQ ID No 3) and 16311G (SEQ ID No 4), without ethidium bromide. Lane 15 through 16: 50 pmole 16311A (SEQ ID No 3) and 16311G (SEQ ID No 4), without ethidium bromide. Lane 17 through 18: 5 pmole 16311A (SEQ ID No 3) and 16311G (SEQ ID No 4), without ethidium bromide. Lane 19 through 20: 2.5 pmole 16311A (SEQ ID No 3), and 16311G (SEQ ID No 4), without ethidium bromide. Lane 21 through 22: 0.5 pmole 16311A (SEQ ID No 3) and 16311G (SEQ ID No 4), without ethidium bromide. Lane 23: 100 bp ladder (Promega Corp.).

FIG. 6 shows the amplification results on mtDNA in 2% agarose gel stained with ethidium bromide showing the amplification results on mtDNA carrying a T to C transition at position 16311 with 5.0 ug/ml ethidium bromide using primers FW I and either 16311A (SEQ ID No 3) or 16311G (SEQ ID No 4) at different concentrations of template.

Lane 1 and 2: 16311A (SEQ ID No 3) and 16311G (SEQ ID No 4) at 500 ng total DNA, 5.0 ug/ml ethidium bromide. Lane 3 and 4: 16311A (SEQ ID No 3) and 16311G (SEQ ID No 4) at 50 ng total DNA, 5.0 ug/ml ethidium bromide. Lane 5 and 6: 16311A (SEQ ID No 3) and 16311G (SEQ ID No 4) at 10 ng total DNA, 5.0 ug/ml ethidium bromide. Lane 7 and 8: 16311A (SEQ ID No 3) and 16311G (SEQ ID No 4) at 2 ng total DNA, 5.0 ug/ml ethidium bromide. Lane 9 and 10: 16311A (SEQ ID No 3) and 16311G (SEQ ID No 4) at 0.5 ng total DNA, 5.0 ug/ml ethidium bromide. Lane 11: 100 bp ladder (Promega Corp.). Lane 13 and 14: 16311A (SEQ ID No 3) and 16311G (SEQ ID No 4) at 500 ng total DNA, without ethidium bromide. Lane 15 and 16: 16311A (SEQ ID No 3) and 16311G (SEQ ID No 4) at 50 ng total DNA, without ethidium bromide. Lane 17 and 18: 16311A (SEQ ID No 3) and 16311G (SEQ ID No 4) at 10 ng total DNA, without ethidium bromide. Lane 19 and 20: 16311A (SEQ ID No 3) and 16311G (SEQ ID No 4) at 2 ng total DNA, without ethidium bromide. Lane 21 and 22: 16311A (SEQ ID No 3) and 16311G (SEQ ID No 4) at 0.5 ng total DNA, without ethidium bromide. Lane 23: 100 bp ladder (Promega Corp.).

FIG. 7 shows the amplification results on mtDNA in 2% agarose gel stained with ethidium bromide showing the amplification results on mtDNA carrying a T to C transition at position 16311 with primers FW I and either 16311A (SEQ ID No 3) or 16311G (SEQ ID No 4) adding 5.0 ug/ml ethidium bromide before, in between and after the downstream primers and the template.

Lane 1 and 2: 16311A (SEQ ID No 3) and 16311G (SEQ ID No 4) with 5.0 ug/ml ethidium bromide added after the primers and the template. Lane 3 and 4: 16311A (SEQ ID No 3) and 16311G (SEQ ID No 4) with 5.0 ug/ml ethidium bromide added after the template and before the primers. Lane 5 and 6: 16311A (SEQ ID No 3) and 16311G (SEQ ID No 4) with 5.0 ug/ml ethidium bromide added before the primers and the primers template. Lane 7 and 8: 16311A (SEQ ID No 3) and 16311G (SEQ ID No 4) with 5.0 ug/ml ethidium bromide added after the primers and before the template. Lane 9: 100 bp ladder (Promega Corp.). Lane 10 and 11: 16311A (SEQ ID No 3) and 16311G (SEQ ID No 4) with water added after the primers and the template. Lane 12 and 13: 16311A (SEQ ID No 3) and 16311G (SEQ ID No 4) with water added after the template and before the primers. Lane 14 and 15: 16311A (SEQ ID No 3) and 16311G (SEQ ID No 4) with water added before the primers and the primers template. Lane 16 and 17: 16311A (SEQ ID No 3) and 16311G (SEQ ID No 4) with water added after the primers and before the template. Lane 18: 100 bp ladder (Promega Corp.).

FIG. 8 shows the amplification results on mtDNA in 2% agarose gel stained with ethidium bromide showing the Pfu DNA Polymerase amplification results on mtDNA carrying a T to C transition at position 16311 using primers FW I (SEQ ID No 1) and either 16311A (SEQ ID No 3), 16311G (SEQ ID No 4), 16311T (SEQ ID No 5) or 16311C (SEQ ID No 6) at different ethidium bromide concentrations.

Lane 1 through 4: 16311A (SEQ ID No 3), 16311G (SEQ ID No 4), 16311T (SEQ ID No 5) and 16311C (SEQ ID NO 6) without ethidium bromide. Lane 5 through 8: 16311A (SEQ ID No 3), 16311G (SEQ ID No 4), 16311T (SEQ ID No 5) and 16311C (SEQ ID No 6) with 4.5 ug/ml ethidium bromide. Lane 9: 100 bp ladder (Promega Corp.). Lane 10 through 13: 16311A (SEQ ID No 3), 16311G (SEQ ID No 4), 16311T (SEQ ID No 5) and 16311C (SEQ ID No 6) with 5.0 ug/ml ethidium bromide. Lane 14 through 17: 16311A (SEQ ID No 3), 16311G (SEQ ID No 4), 16311T (SEQ ID No 5) and 16311C (SEQ ID No 6) with 5.5 ug/ml ethidium bromide. Lane 18: 100 bp ladder (Promega Corp.).

FIG. 9 shows the amplification results on genomic DNA from human patient carrying a C to T transition at position 1785 of the exon 8 of Homo sapiens cytochrome b-245 beta polypeptide (CYBB) gene and from an individual control in 2% agarose gel stained with ethidium bromide. DNA samples were amplified with or without ethidium bromide in four separate reactions sharing the upstream primer CYBB8FW (SEQ ID No 15) and having either of the four alternative downstream primers (CYBB8A (SEQ ID No: 16), CYBB8G (SEQ ID N°: 17), CYBB8T (SEQ ID No: 18) and CYBB8C (SEQ ID N°: 19)).

Lane 1 through 4: CYBB8A (SEQ ID No: 16), CYBB8G (SEQ ID No: 17), CYBB8T (SEQ ID No: 18) and CYBB8C (SEQ ID N°: 19) without ethidium bromide, patient DNA. Lane 5 through 8: CYBB8A (SEQ ID No: 16), CYBB8G (SEQ ID N°: 17), CYBB8T (SEQ ID No: 18) and CYBB8C (SEQ ID No: 19) without ethidium bromide, control DNA. Lane 9: 100 bp ladder (Promega Corp.). Lane 10 through 13: CYBB8A (SEQ ID No: 16), CYBB8G (SEQ ID No: 17), CYBB8T (SEQ ID No: 18) and CYBB8C (SEQ ID No: 19) with 5.0 ug/ml ethidium bromide, patient DNA. Lane 14 through 17: CYBB8A (SEQ ID No: 16), CYBB8G (SEQ ID No: 17), CYBB8T (SEQ ID No: 18) and CYBB8C (SEQ ID No: 19) with 5.0 ug/ml ethidium bromide, control DNA. Lane 18: 100 bp ladder (Promega Corp.)

### DESCRIPTION OF THE PREFERRED EMBODIMENTS

Definitions:

As used herein, the term “intercalating agent” refers a moiety that is able to intercalate between the bases of a nucleic acid molecule.

As used herein, the term “transition” is the substitution in DNA or RNA of one purine by another purine, or of one pyrimidine by another pyrimidine.

The present invention provides methods for detecting and identifying sequence variation in a nucleic acid by primer extension of polymerases in the presence of an intercalating agent as ethidium bromide when the 3′ end of a primer does not hybridized perfectly with the target sequence. The method can be adapted for use as a means for distinguishing or identifying the nucleotide in the target sequence, which is at the site where the mismatch between the primer and the target occurs.

The efficiency of primer extension is detected as an indication of the presence and/or identity of the sequence variation in the target. The methods are particularly well suited for detecting and identifying single nucleotide differences between a target sequence of interest, for example a mutant allele of the gene, and a second nucleic acid sequence, for example a wild type allele for the same gene.

Addition of the intercalating agent for example ethidium bromide to an extension reaction mixture or hybridization medium, preferably a PCR reaction mixture improves the selectivity of sequence-specific analyses by decreasing 3′-mismatch priming. The methods of the invention increase the selectivity of gene variation analyses and suppress the formation of false positive to prevent wrong diagnoses and findings. The method of the invention may be used in any assay that employs an extension step of a target nucleic acid sequence, wherein the 3′ en of the primer does not hybridized perfectly with that target nucleic acid sequence.

The extension reaction mixture or hybridization mediums can be any conventional medium known to be suitable for the extension reaction, for example the liquid medium can comprise nucleotide sequence, primers, water, buffer and salts. The extension reaction can be carry out under a wide variety of conditions, having different temperature, electrostatic strength, salt concentration and composition.

It is known that the concentration of the intercalating agent must be adjusted according the extension conditions and the type of intercalating agent used. In a preferred embodiment, the intercalating agent is ethidium bromide, which is present in the extension reaction mixture in a concentration about 4 μg/ml and 7 μg/ml, preferably 5 μg/ml in a PCR.

The use of the inventive reaction mixture leads to a clearly improved sensitivity and selectivity of gene polymorphism and gene mutation analyses in animal, bacterial, plants and human genome, preventing wrong diagnoses. By these means, it is possible to carry out such detections on samples, which previously could not be analyzed in this way. Moreover, the invention leads to a dramatic reduction in the costs of detections, and is rapid and sensitive.

The inventive extension reaction mixture makes possible a distinct increase in the information power of the semi-quantitative and totally quantitative determination of gene variation in tissues and organs in healthy, diseased and medicinally affected state.

In a preferred embodiment, the method of the invention is directed to detecting single nucleotides polymorphisms (SNPs) in a nucleic acid sequence of interest, for example alleles, and to identifying such SNPs or alleles. Such nucleotide sequence variants may be detected directly in a sample to be analyzed during extension and/or amplification of the target sequence.

The inventive methods are based upon de relative inefficiency of primer extension by polymerases enzymes in the presence of an intercalating agent when there are mismatches at the 3′ end of a primer hybridized to an otherwise complementary sequence. The method of the invention is useful for detecting mismatches at the 3′ end when purine-pyrimidine, purine-purine or pyrimidine-pyrimidine bases mismatches are present.

The difference in the efficiency of polymerase extension (in presence of ethidium bromide) when the primer is hybridized to two different alleles may be used to indicate which allele the target nucleic acid contains. When any one of multiple alleles may be present, multiple primers are employed in the analysis, each with different potential mismatch at or near 3′ end. The primer which is most efficient extended provides the identity of the allele, for example the identity of the nucleotide present in the target sequence being analyzed. If the set of primers comprising A, G, C and T at the site of allele to be identified is hybridized to the target sequence and extended, the identity of the allele will be the complement of the nucleotide in the signal primer which was most efficiently extended by the polymerase. The reaction may be performed in monoplex or multiplex format, containing either one or more sets of allelic primers.

The present invention is suitable for SNP assays and for variations that involve mismatches larger than one nucleotide. It may be applied to the currently used methods that rely on primer annealing to distinguish variations in nucleic acid. It may also be useful in the design of defined or random approaches to discover new SNPs.

The application of the invention comprises, above all, a) the pharmacogenomics, especially the discovery of genomic target for drug candidates, b) detection of nucleotide polymorphisms, especially in molecular diagnosis of disease based on gene mutation analyses and gene polymorphisms, c) molecular diagnosis, especially the screening and diagnosis of illness relevant genes.

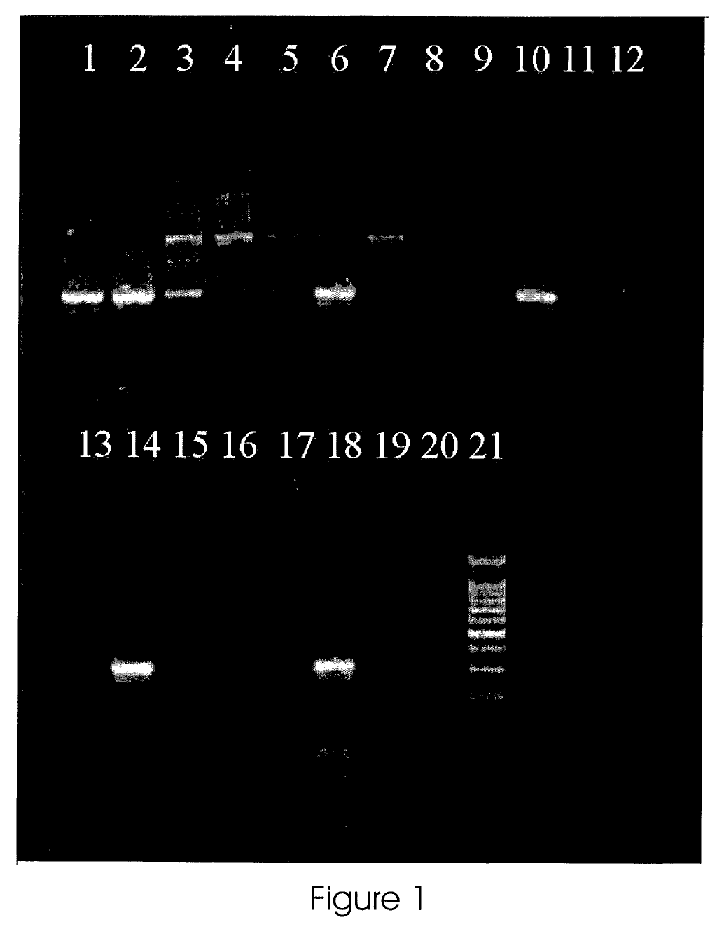

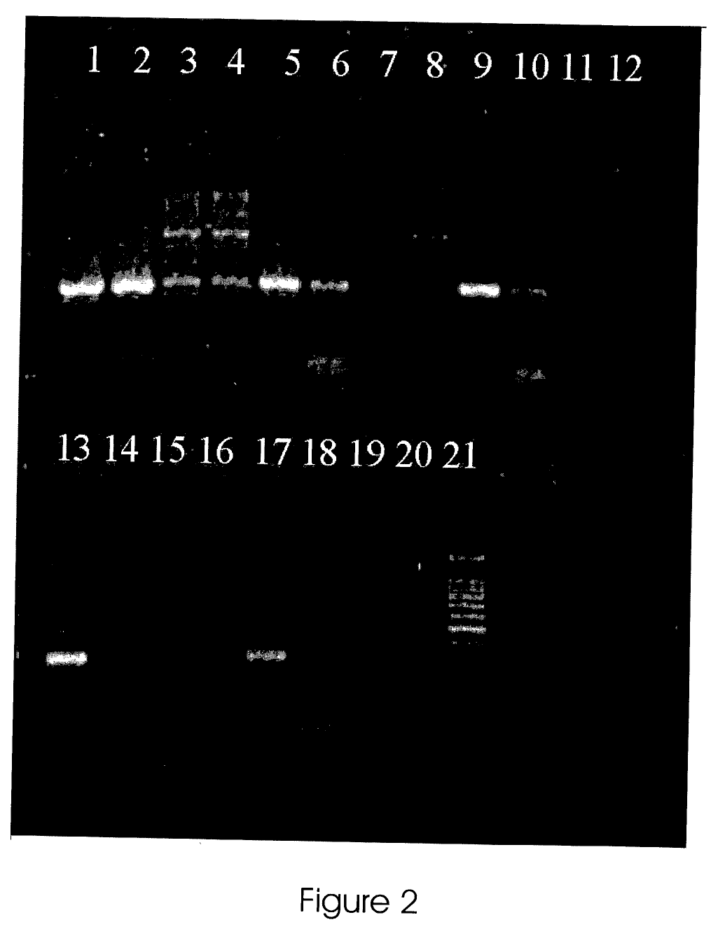

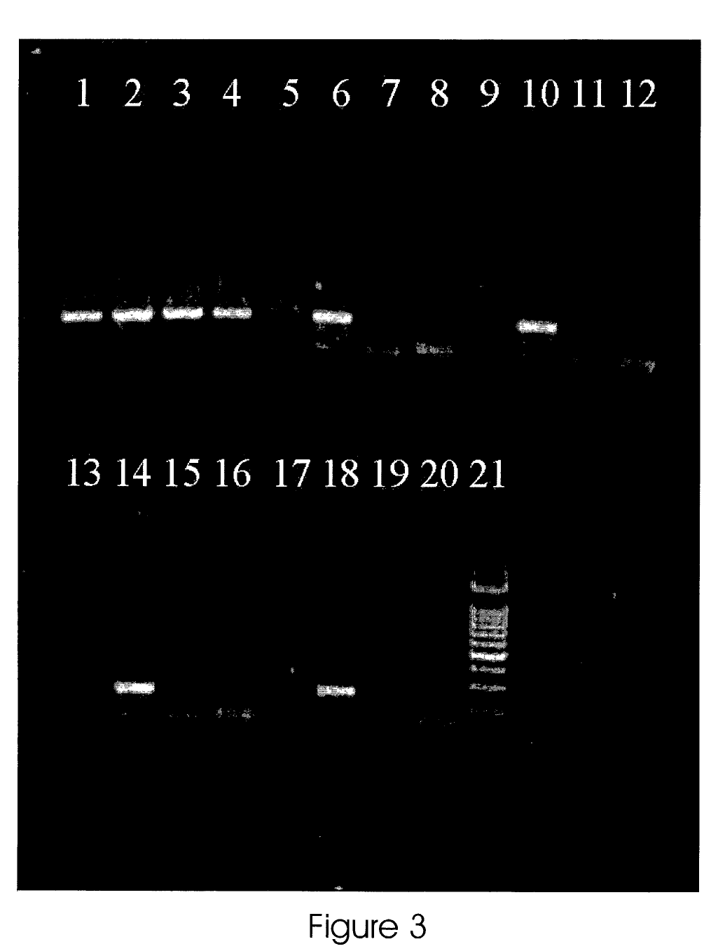

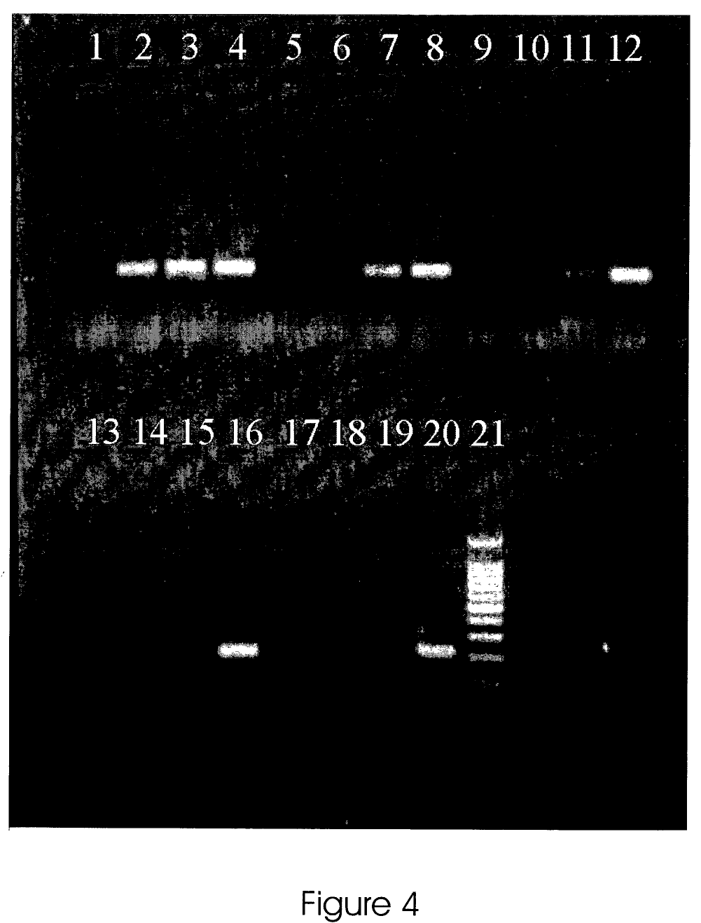

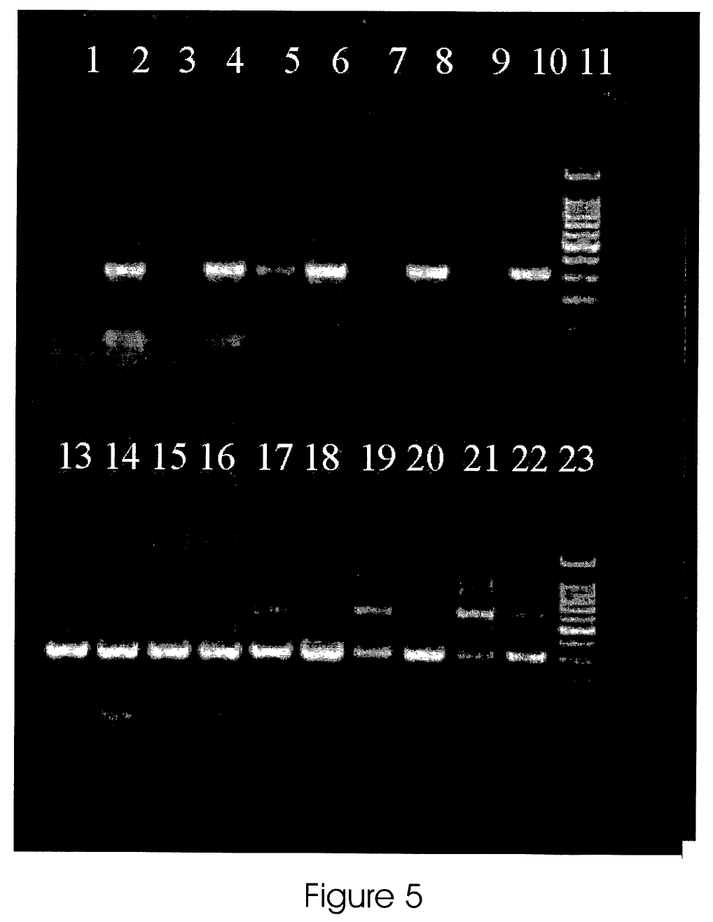

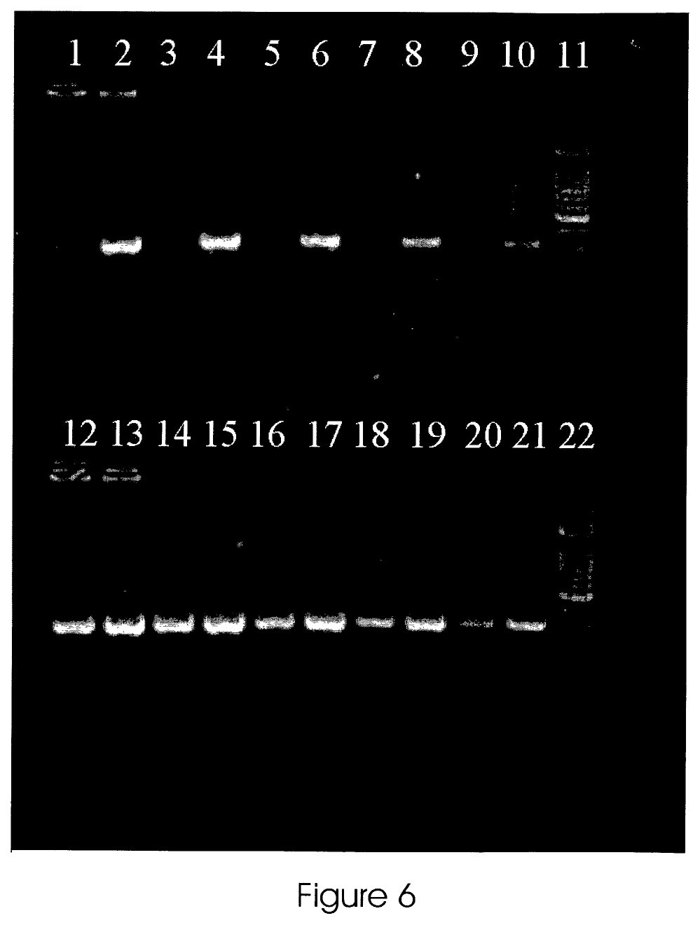

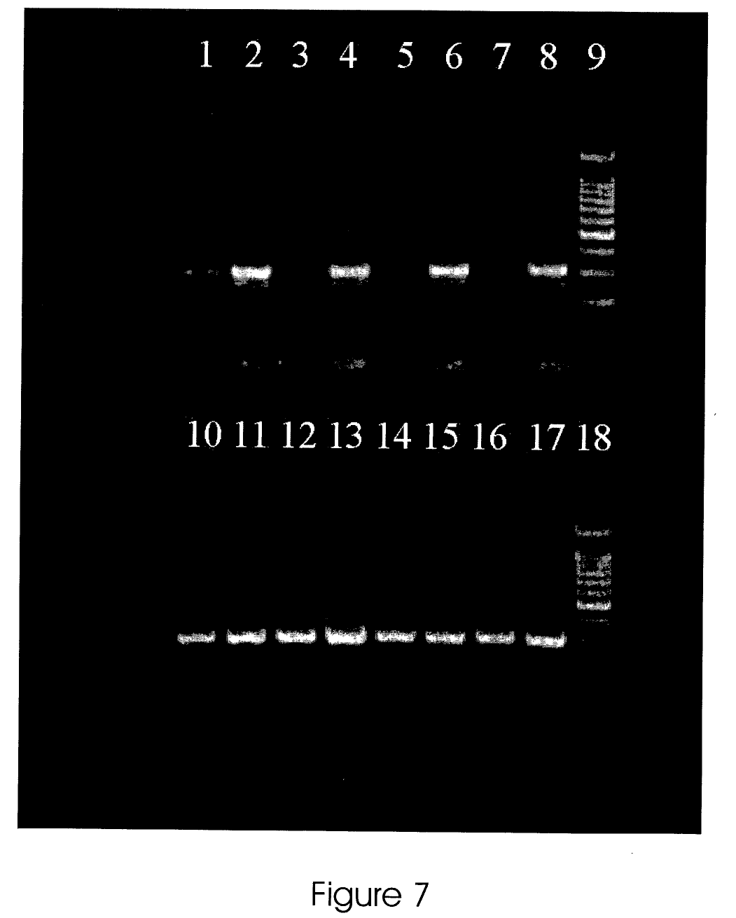

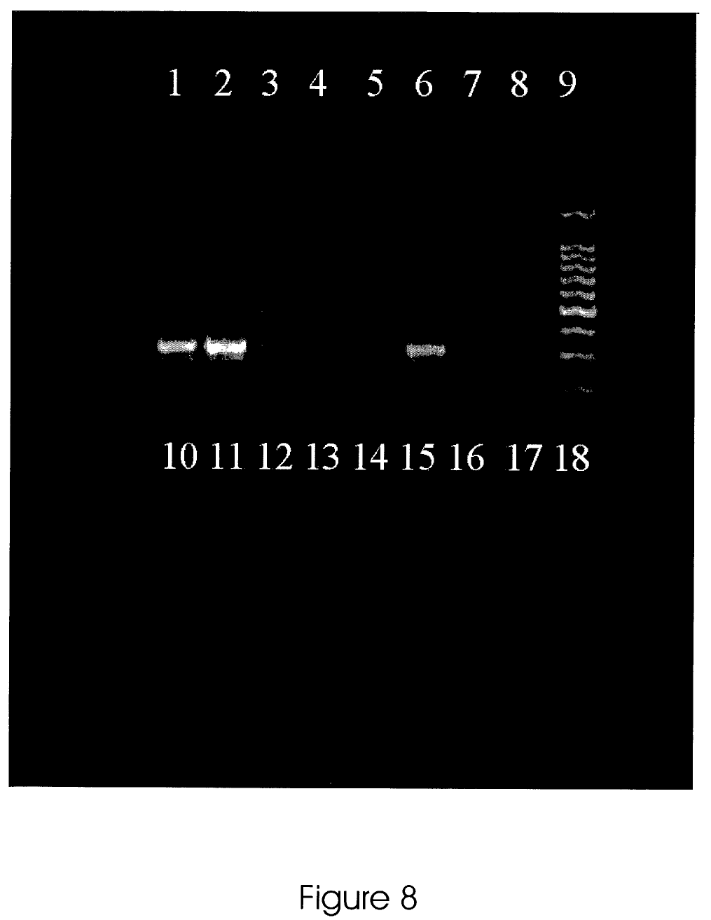

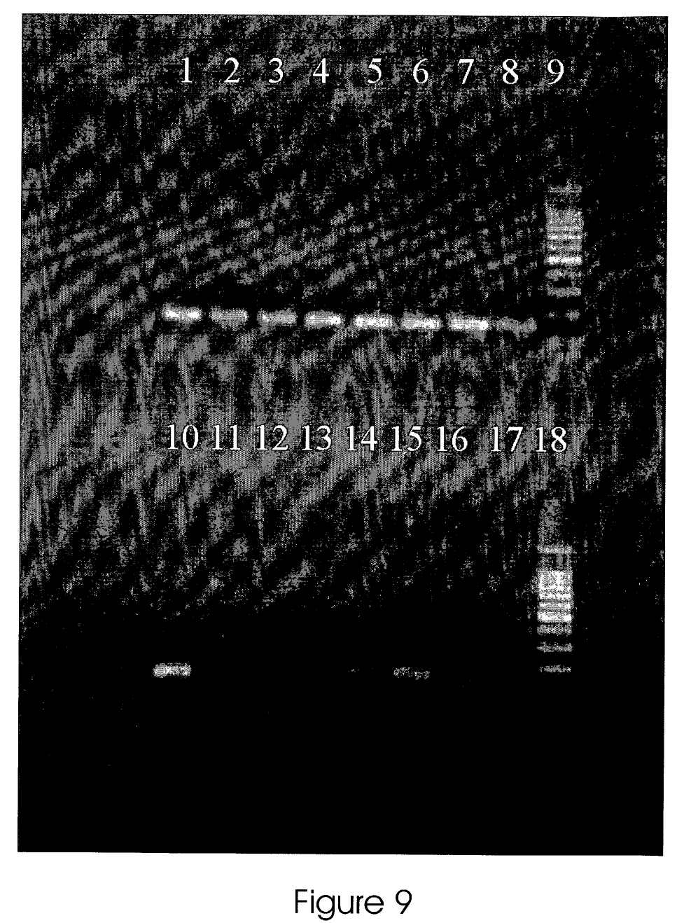

The inventors demonstrated that the addition of EtBr improves the specificity of DNA amplification when a 299 bp region of the human mitochondrial D-loop (16,031-16,330) carrying cytosine nucleotide (C) at position 16311 was amplified in four separate PCR reactions sharing the same upstream primer (FW I (SEQ ID No 1)) and having either of the four 3′-end alternative downstream primers (16311A (SEQ ID No 3), 16311G (SEQ ID No 4), 16311T (SEQ ID No 5) and 16311C (SEQ ID No 6)). Increasing concentrations of ethidium bromide were added to assess the effect on the amplification of the primers carrying different 3′ nucleotides. In the absence of ethidium bromide there were ambiguous results. Mismatch primer 16311A (SEQ ID No 3) yielded about the same amount of product as the fully homologous primer 16311G (SEQ ID No 4). There was also some product with mismatch primers 16311T (SEQ ID No 5) and 16311C (SEQ ID No 6). The addition of ethidium bromide at concentrations ranging from 4.5 ug/ml to 6.0 ug/ml dramatically diminished the non-specific amplification of all 3′ end mismatch primers (16311A (SEQ ID No 3), 16311C (SEQ ID No 6) and 16311T (SEQ ID No 5)), although had little effect on the totally complementary one (16311G (SEQ ID No 4)) (FIG. 1). Reciprocally, the same good discrimination was obtained with primer 16311A (SEQ ID No 3) using mtDNA from a donor carrying Anderson's consensus nucleotide thymine (T) instead of cytosine (C) at position 16311 (12) (FIG. 2). All mtDNA sequences were confirmed by DNA sequencing.

In order to verify if the effect of ethidium bromide was similar in other amplifications the inventors tested other regions of mtDNA. They amplified a 244 bp fragment of region I (16,031-16,275) using upstream primer FW I and downstream primers 16256A (SEQ ID No 7), 16256G (SEQ ID No 8), 16256T (SEQ ID No 9) and 16256C (SEQ ID No 10) and a 219 bp fragment of region II (16513-162) using upstream primer FW II and downstream primers 143A (SEQ ID No 11), 143G (SEQ ID No 12), 143T (SEQ ID No 13) and 143C (SEQ ID No 14). Again, the non-specific amplification of mismatch primers was abolished in all cases, regardless of the sequence being amplified (FIG. 3 and FIG. 4). As shown in FIG. 4 some small variations around the optimal concentration of ethidium bromide are observed, which may be due to the different sequences of the primers.

To assess if the primer concentration can affect the selectivity brought about by the addition of ethidium bromide, the inventors tested different amounts of the otherwise hard to distinguish primers 16311A (SEQ ID No 3) and 16311G (SEQ ID No 4) in mtDNA carrying cytosine nucleotide (C) at position 16311. No difference in the results was found at the primer amount range tested (0.5, 2.5, 5.0, 50 and 100 pmole) (FIG. 5).

To verify if ethidium bromide was effective with different amounts of template, the inventors added different amounts of mtDNA carrying cytosine nucleotide (C) at position 16311 to the reaction containing either primer 16311A (SEQ ID No 3) or 16311G (SEQ ID No 4). The selectivity remained the same regardless of the amount of DNA template used (0.5, 2.0, 10.0, 50 and 500 ng) (FIG. 6).

In order to asses if it was necessary to add the intercalating agent before the primer and the template got together in the reaction, the inventors performed PCR amplifications adding the ethidium bromide before, in between and after the addition of the primer and the template. No differences were observed in any case (FIG. 7).

The inventors tested the effect of ethidium bromide effect when a DNA polymerase carrying a proof-reading, 31-5′ exonuclease activity was used in the reaction instead of Taq DNA Polymerase. The 299 bp region of the mtDNA (16,031-16,330) carrying cytosine nucleotide (C) at position 16311 was amplified with Pfu DNA Polymerase (Promega) in four separate PCR reactions using primer FW I (SEQ ID No 1) and the four alternative downstream primers (16311G (SEQ ID No 4), 16311A (SEQ ID No 3), 16311C (SEQ ID No 6) and 16311T (SEQ ID No 5)) at different ethidium bromide concentrations. Surprisingly, despite the editing activity of Pfu DNA Polymerase, it was obtained good specific results at a concentration range of ethidium bromide similar to the one of Taq (FIG. 8). The invention does not require a specific polymerase, any polymerase may be used to obtain the suitable product in an extension reaction.

The inventors tested the discriminatory effect of ethidium bromide on genomic DNA by amplifying a 153 bp region of the exon 8 of Homo sapiens cytochrome b-245 beta polypeptide (CYBB) gene (AH011465). CYBB mutations are involved in the X-linked chronic granulomatous disease (CGD) (Jirapongsananuruk, O., et al., Clin. Immunol. 104: 73, 2002, cited herein as references). DNA from a male patient having a C to T transition at nucleotide position 1785 and DNA from a male control individual were amplified with or without ethidium bromide in four separate reactions sharing the upstream primer CYBB8FW (SEQ ID No 15) and having either of the four alternative downstream primers (CYBB8A (SEQ ID NO 16), CYBB8G (SEQ ID NO 17), CYBB8T (SEQ ID NO 18) and CYBB8C (SEQ ID NO 19)). Reactions without ethidium bromide gave a non-specific allele amplification product with the four primers in both the patient and the control. On the other hand, reactions containing ethidium bromide at 5 ug/ml yielded a distinct amplification band only with their corresponding homologous primers, CYBBA (SEQ ID No 16) in the patient and CYBBG (SEQ ID No 17) in the control (FIG. 9). This result shows that the method of the invention may be used when the template is genomic DNA.

The method of the invention can be used employing any template sequences in an extension reactions wherein the template is genomic DNA, mitochondrial DNA, synthetic DNA or any nucleotide sequence as RNA sequences when the 3′ end of the primer does not hybridized correctly with template target sequence.

Throughout this application, various publications are referenced. The disclosures of all of theses publications and those references are hereby incorporated by reference into this application in order to more fully describe the state of the art to which this invention pertains.

It should also be understood that the foregoing relates to preferred embodiments of the present invention and that numerous changes may be made therein without departing from the scope of the invention. The invention is further illustrated by the following examples, which are not to be construed in any way as imposing limitation upon the scope thereof. On the contrary, it is to be clearly understood that resort may be had to various other embodiments, modifications, and equivalents thereof, after reading the description herein, may suggest themselves to those skilled in the art without departing from the spirit of the present invention and/or the scope of the appended claims.

### EXAMPLES

Example 1

### DNA Purification and PCR Amplification

Total DNA was purified from fresh human blood by a salting out procedure using the Wizard® Genomic DNA Purification kit (Promega). Blood was collected in 1.5 ml microtubes containing 100 ul 0.5M EDTA as anticoagulant. Total yield of DNA was about to 10 ug for each sample.

PCR amplifications were performed in a MJ Research PTC-150 thermal cycler in a 25 ul reaction volume containing 10 mM Tris-HCl (pH 9.0), 50 mM KCl, 0.1% Triton X-100, 1.5 mM MgCl2, 200 uM each deoxy-NTP, 1.5 U Taq DNA Polymerase (Promega Corp.), 25 pmol of upstream primer, 10 pmol of downstream primer and 10 ng of total DNA. Cycling conditions included an initial denaturation step of 2 min at 95° C., followed by 36 cycles of 30 seg at 95° C., 30 seg at 53° C. and 1 min at 72° C. PCR products were electrophoresed through a 2% agarose gel and visualized with ethidium bromide.

### Example 2

### Addition of EtBr to the PCR Reaction Mixture to Improve the Specificity of DNA Amplification

A 299 bp region of the human mitochondrial D-loop (16,031-16,330) carrying cytosine nucleotide (C) at position 16311 was amplified in four separate PCR reactions sharing the same upstream primer (FWD I Upstream primer 5′-ATG GGG AAG CAG ATT TGG GT-31 (SEQ ID No 1)) and having either of the four 3′-end alternative downstream primers (16311A (SEQ ID No 3) downstream primer 5′-ACG GTA AAT GGC TTT ATG TA-3′, 16311G (SEQ ID No 4) downstream primer 5′-ACG GTA AAT GGC TTT ATG TG-3′, 16311T (SEQ ID No 5) downstream primer 5′-ACG GTA AAT GGC TTT ATG TT-3′, 16311C (SEQ ID No 6) downstream primer 5′-ACG GTA AAT GGC TTT ATG TC-3′). The PCR was carry out as in the example 1, except of increasing concentrations of ethidium bromide were added to assess the effect on the amplification of the primers carrying different 3′ nucleotides. The increasing concentrations of ethidium bromide were from 4.5 to 6.0 μg/ml.

In order to verify if the effect of ethidium bromide was similar in other amplifications, we tested other regions of mtDNA. We amplified a 244 bp fragment of region I (16,031-16,275) using upstream primer FW I (showed above) and downstream primers (16256A (SEQ ID No 7) downstream primer 5′-TCC TAG TGG GTG AGG GGT GA-3′, 16256G (SEQ ID No 8) downstream primer 5′-TCC TAG TGG GTG AGG GGT GG-3′, 16256T (SEQ ID No 9) downstream primer 5′-TCC TAG TGG GTG AGG GGT GT-3′ and 16256C (SEQ ID No 10) downstream primer 5′-TCC TAG TGG GTG AGG GGT GC-3′); and a 219 bp fragment of region II (16513-162) using upstream primer FW II (FWD II Upstream primer 5′-TCA GGG TCA TAA AGC CTA AA-3′(SEQ ID No 2)) and downstream primers 143A (SEQ ID No 11) downstream primer 5′-GAT AAA TAA TAG GAT GAG GA-3′, 143G (SEQ ID No 12) downstream primer 5′-GAT AAA TAA TAG GAT GAG GG-3′, 143T (SEQ ID No 13) downstream primer 5′-GAT AAA TAA TAG GAT GAG GT-3′, 143C (SEQ ID No 14) downstream primer 5′-GAT AAA TAA TAG GAT GAG GC-3′).

The PCR was carry out as in the example 1, except of increasing concentrations of ethidium bromide were added to assess the effect on the amplification of the primers carrying different 3′ nucleotides in different regions of mtDNA. The increasing concentrations of ethidium bromide were from 4.5 to 6.0 μg/ml in the amplification of the 244 bp fragment and from 5.0 to 6.5 μg/ml in the amplification of the 219 bp fragment.

### Example 3

### The Effect of Primer Concentration and Amount of Template in the Methods of the Invention

To assess if the primer concentration can affect the selectivity brought about by the addition of ethidium bromide, was tested different amounts of the otherwise hard to distinguish primers 16311A (SEQ ID No 3) and 16311G (SEQ ID No 4) (sequence showed above) in mtDNA carrying cytosine nucleotide (C) at position 16311. The PCR was carried out as in the example 1. Ethidium bromide concentration in the PCR reaction mixture was 5.0 μg/ml and primers amounts were from 0.5 to 100 pmole.

To verify if ethidium bromide was equally effective with different amounts of template, were added different amounts of mtDNA carrying cytosine nucleotide (C) at position 16311 to the reaction containing either primer 16311A (SEQ ID No 3) or 16311G (SEQ ID No 4).

The PCR was carried out as in the example 1. Ethidium bromide concentration in the PCR reaction mixture was 5.0 μg/ml, template amount was from 0.5 ng to 500 ng.

### Example 4

### Adding Ethidium Bromide at Different Time

In order to asses if it was necessary to add the intercalating agent before the primer and the template got together in the reaction, we performed PCR amplifications adding the ethidium bromide before, in between and after the addition of the primer and the template.

The PCR was carried out as in the example 1 adding 5.0 μg/ml of ethidium bromide before, between and after the primers and template.

### Example 5

### PCR Amplification Using Proof Reading Enzyme Instead Tag DNA Polimerase

PCR reaction mixture and conditions:

Proof-reading amplifications were performed with 0.6 U of Pfu DNA Polymerase (Promega Corp.) in 20 mM Tris-HCl (pH 8.8), 10 mM KCl, 0.1% Triton® X-100, 2 mM MgSO4, 10 nM (NH4)SO₄, 0.1 mg/ml BSA, 200 uM each deoxy-NTP, 25 pmol of upstream primer, 10 pmol of downstream primer and 10 ng of total DNA under the same cycling conditions described in the example 1, except of increasing concentrations of ethidium bromide (from 4.5 to 5.5 μg/ml) were added. PCR products were electrophoresed through a 2% agarose gel and visualized with ethidium bromide.

Primers and templates:

The 299 bp region of the mtDNA (16,031-16,330) carrying cytosine nucleotide (C) at position 16311 was amplified with Pfu DNA Polymerase (Promega) in four separate PCR reactions using primer FW I (sequence showed above) and the four alternative downstream primers 16311G (SEQ ID No 4), 16311A (SEQ ID No 3), 16311C (SEQ ID No 6) and 16311T (SEQ ID No 5) (sequences showed above).

### Example 6

### Detection of a Single-Base Mutation in Exon 8 of Homo Sapiens Cytochrome b-245 Beta Polypeptide (CYBB) Gene Using the Method of the Invention

The PCR was carried out as in the example 1, adding 10 pmole of both primers and 5.0 μg/ml of ethidium bromide. The sequence of the primers was:

| | | |
|---|---|---|
| CYBB8FW Upstream primer | 5′-CTC CCT CTG AAT ATT TTG TTA TC-3′ | (SEQ ID No 15) |
| CYBB8A Downstream primer | 5′-GAC CAC CTT CTG TTG AGA TCA-3′ | (SEQ ID No 16) |
| CYBB8G Downstream primer | 5′-GAC CAC CTT CTG TTG AGA TCG-3′ | (SEQ ID No 17) |
| CYBB8T Downstream primer | 5′-GAC CAC CTT CTG TTG AGA TCT-3′ | (SEQ ID No 18) |
| CYBB8C Downstream primer | 5′-GAC CAC CTT CTG TTG AGA TCC-3′ | (SEQ ID No 19) |

While preferred embodiments of the present invention have been illustrated and described, it will be obvious to those skilled in the art that various changes and modifications may be made therein without departing from the scope of the invention as defined in the appended claims.

We claim:

1. A method for reducing primer extension by polymerase enzymes when the 3′ end of a primer does not hybridize perfectly with the target, increasing the selectivity of single nucleotide mutation or gene analyses by suppressing false positive results, comprising the steps of:
(a) obtaining a nucleic acid sample;
(b) hybridizing said nucleic acid sample to a primer;
(c) subjecting said nucleic acid sample hybridized to an extension reaction by extending the primer with a polymerizing enzyme, wherein the reaction extension mixture medium contains an intercalating agent;
(d) detecting the presence of extension products.

2. The method of claim 1, wherein the step (c) in the extension reaction by extending a primer with a polymerizing enzyme is the extension reaction steps in a PCR procedure and the extension reaction medium is a PCR reaction mixture, wherein said PCR reaction mixture contains an intercalating agent and, the step (d) is the detection of amplified products.

3. The method of claim 1 wherein the intercalating agent is a flat organic molecule capable of stacking between the nucleic acid bases.

4. The method of claim 3, wherein the intercalating agent is selected from the group consisting of ethidium bromide, dihydroethidium, ethidium homodimer-1, ethidium homodimer-2, acridine, propidium iodide, YOYO®-1 and TOTO®-1.

5. The method of claim 3, wherein the intercalating agent is ethidium bromide.

6. The method of claim 2, wherein the intercalating agent concentration is about 4 to 7 μg/ml in the PCR reaction mixture.

7. The method of claim 1, wherein the step (b) comprises hybridized the nucleic acid sample to primers in a monoplex or multiplex format containing either one or more sets of allelic primers.

8. The method of claim 2, wherein one or more primers is/are employed in the PCR procedure.

9. The method of claim 1, wherein polymerizing enzyme is selected from the group consisting of proof-reading DNA polymerases and DNA or RNA polymerases, with or without editing activity, native or recombinant, thermostable or non-thermostable, complete enzyme or some active fragment.

10. The method of claim 1, wherein the presence of ethidium bromide in the extension reaction (1) decrease the amount of extension products when the 3′ end of a primer does not hybridize perfectly with the target sequence, (2) increase the selectivity detection of single nucleotide mutation and/or (3) suppresses false positive extension products in gene analyses.

11. A mixture reagent for an extension reaction, wherein the mixture reagent comprises:
a polymerizing enzyme, dNTPs, a buffer, an intercalating agent, primers, template and salts.

12. The mixture reagent of claim 11, wherein the intercalating agent is a flat organic molecule capable of stacking between the nucleic acid bases.

13. The mixture reagent of claim 12, wherein the intercalating agent is selected from the group consisting of ethidium bromide, dihydroethidium, ethidium homodimer-1, ethidium homodimer-2, acridine, propidium iodide, YOYO®-1 and TOTO®-1.

14. The mixture reagent of claim 12, wherein the intercalating agent is ethidium bromide and de concentration of ethidium bromide is about 4 to 7 μg/ml.

15. The mixture reagent of claim 11, wherein said mixture reagent is a PCR reaction mixture.

16. The mixture reagent of claim 11, wherein polymerizing enzyme is selected from the group consisting of proof-reading ADN polymerases and DNA or RNA polymerases, with or without editing activity, native or recombinant, thermostable or non-thermostable, complete enzyme or some active fragment.


SEQUENCE LISTING (19 sequences)

SEQ ID NO: 1 - DNA, 20 bp, Artificial
  Human mitochondrial D-loop (16,031-16,330, carrying cytosine nucleotide (C) at position 16311) upstream primer
atggggaagcagatttgggt

SEQ ID NO: 2 - DNA, 20 bp, Artificial
  Human mtDNA (219 bp fragment of region II, 16513-162) upstream primer
tcagggtcataaagcctaaa

SEQ ID NO: 3 - DNA, 20 bp, Artificial
  Human mitochondrial D-loop (16,031-16,330, carrying adenosine nucleotide (A) at position 16311) downstream primer
acggtaaatggctttatgta

SEQ ID NO: 4 - DNA, 20 bp, Artificial
  Human mitochondrial D-loop (16,031-16,330, carrying guanosine nucleotide (G) at position 16311) downstream primer
acggtaaatggctttatgtg

SEQ ID NO: 5 - DNA, 20 bp, Artificial
  Human mitochondrial D-loop (16,031-16,330, carrying thymidine nucleotide (T) at position 16311) downstream primer
acggtaaatggctttatgtt

SEQ ID NO: 6 - DNA, 20 bp, Artificial
  Human mitochondrial D-loop (16,031-16,330, carrying cytosine nucleotide (C) at position 16311) downstream primer
acggtaaatggctttatgtc

SEQ ID NO: 7 - DNA, 20 bp, Artificial
  Human mtDNA (244 bp fragment of region I, 16,03 1-16,275, carrying adenosine nucleotide (A) at position 16256) downstream primer
tcctagtgggtgaggggtga

SEQ ID NO: 8 - DNA, 20 bp, Artificial
  Human mtDNA (244 bp fragment of region I, 16,03 1-16,275, carrying guanosine nucleotide (G) at position 16256) downstream primer
tcctagtgggtgaggggtgg

SEQ ID NO: 9 - DNA, 20 bp, Artificial
  Human mtDNA (244 bp fragment of region I, 16,03 1-16,275, carrying thyrosine nucleotide (T) at position-16256) downstream primer
tcctagtgggtgaggggtgt

SEQ ID NO: 10 - DNA, 20 bp, Artificial
  Human mtDNA (244 bp fragment of region I, 16,03 1-16,275, carrying cytosine nucleotide (C) at position 16256) downstream primer
tcctagtgggtgaggggtgc

SEQ ID NO: 11 - DNA, 20 bp, Artificial
  Human mtDNA downstream primer
gataaataataggatgagga

SEQ ID NO: 12 - DNA, 20 bp, Artificial
  Human mtDNA downstream primer
gataaataataggatgaggg

SEQ ID NO: 13 - DNA, 20 bp, Artificial
  Human mtDNA downstream primer
gataaataataggatgaggt

SEQ ID NO: 14 - DNA, 20 bp, Artificial
  Human mtDNA downstream primer
gataaataataggatgaggc

SEQ ID NO: 15 - DNA, 23 bp, Artificial
  Homo sapiens Cytochrome b-245 beta upstream primer
ctccctctgaatattttgttatc

SEQ ID NO: 16 - DNA, 21 bp, Artificial
  Homo sapiens Cytochrome b-245 beta downstream primer
gaccaccttctgttgagatca

SEQ ID NO: 17 - DNA, 21 bp, Artificial
  Homo sapiens Cytochrome b-245 beta downstream primer
gaccaccttctgttgagatcg

SEQ ID NO: 18 - DNA, 21 bp, Artificial
  Homo sapiens Cytochrome b-245 beta downstream primer
gaccaccttctgttgagatct

SEQ ID NO: 19 - DNA, 21 bp, Artificial
  Homo sapiens Cytochrome b-245 beta downstream primer
gaccaccttctgttgagatcc